(19) United States
(12) Patent Application Publication
(10) Pub. No.: US 2014/0222102 A1
(43) Pub. Date: Aug. 7, 2014

(54) SMART PHONE APPLICATION FOR PROVIDING NEURO/MUSCULAR ELECTRO-STIMULATION

(71) Applicants: ANTHONY LEMUS, VILLA PARK, CA (US); ANDREW LEMUS, VILLA PARK, CA (US); Vincent Lemus, Villa Park, CA (US)

(72) Inventors: ANTHONY LEMUS, VILLA PARK, CA (US); ANDREW LEMUS, VILLA PARK, CA (US); Vincent Lemus, Villa Park, CA (US)

(21) Appl. No.: 13/834,566

(22) Filed: Mar. 15, 2013

Related U.S. Application Data
(60) Provisional application No. 61/761,599, filed on Feb. 6, 2013.

(51) Int. Cl. A61N 1/372; A61N 1/36

(52) U.S. Cl. CPC A61N 1/37235; A61N 1/36003
USPC 607/48

(57) ABSTRACT

An application downloadable to a mobile device is provided for facilitating muscle therapy, the applicable programmed and configurable to generate waveform signals, the waveform signals configured to be employed by a power circuit to generate energy, conforming to the signals, to a muscle pad. The application may be combined as a system with a muscle pad electrically interfacing with the downloadable application, as well as a discrete device in electrical communication with the mobile device and the muscle pad. A power circuit and a muscle metric feedback circuit are contemplated as part of embodiments of a system or kit.

This application claims priority to provisional patent application U.S. Ser. No. 61/761,599 filed on Feb. 6, 2013, the entire contents of which is herein incorporated by reference.

### BACKGROUND

The embodiments herein relate generally to a system for providing neuro-muscular electro-stimulation (i.e., neuro stimulation and/or muscle stimulation) to a stimulator patch adhered to body tissue, and more particularly to the use of a consumer electronic device, such as a smart phone, to control the delivery

The human brain sends electrical stimulus to muscular tissues to contract and expand based on body demands. For instance, athletes place extreme demand on muscular activity to meet the competitive rigors in sports such as running, biking, football, etc. However, through an athlete's career muscle injuries occur and the participant is “sidelined” until the muscular motion can be optimized again. During recovery periods, injury and post activity, the muscles require stimulation to heal and/or tone, and the athlete begins seeking the quickest path to achieve recovery healing, toning, and conditioning. Electro-stimulation is also used in physical therapy applications with prescribed treatment times and power settings.

Electro-stimulation provides a form of therapy through the use of attached electrodes to a site of injury or strengthening and simulates the brain's bio-electro action artificially. Referring to FIGS. 1 and 2, examples of electro-stimulation pads (electrodes) applied to a patient's muscles are shown. The generator unit associated with the electro-stimulation pads, see FIG. 2 for example, sends a voltage/current waveform into the electrode and creates the stimulus for muscular elongation and contraction. FIG. 3 schematically illustrates how differing signals can result in elongation and contraction of muscle tissue. When electrical signals are delivered by the smart phone based system, the muscle will either elongate or contract from its nominal size based on the polarization scheme of the waveform. FIG. 3 shows the muscular displacement synchronized with the signal polarity using a sinusoidal waveform, in this example. The signal amplitude is controlled in conjunction with its frequency and phase angle using a smart phone program similar to a digital signal processor (DSP).

The treatment is applied until muscular healing and re-toning occurs. In theory, artificial muscular activity occurs when the stimulated muscle action activates the creation of nitric oxide (NO), initiating blood vessel dilation. When blood vessels dilate it increases the blood flow containing increased oxygen and nutrients to repair or strengthen tissue allowing for waste products to be extricated. Repetitive application enables therapeutic effects for the user.

Other applications of electro-stimulation include muscle toning and post activity recovery. The principles for these actions are similar. In muscle toning applications, the muscle undergoes artificial stress and strain conditions enhancing its endurance. For post activity, electro-stimulation action promotes the removal of lactic acid and other waste products from the tissue faster.

Today, communication devices have more computing power than early medical electro-surgery equipment. For instance, the Valleylab Force FX electro-surgical generator, required CPU cores and firmware to achieve the output waveforms needed for tissue sealing and cutting in the radio frequency spectrum. The applied output is programmable and incorporates impedance feedback based on a tissue conditions guiding a surgical decisions by the user. Electro-surgery technology can be duplicated in electro-stimulation providing the user with advanced control options for optimized treatment.

In electro-stimulation applications, the voltage and current requirements can be generated with low watt batteries driving a charge pump circuit. For instance, an analog circuit using an old fashion 555 Integrated circuit timer can generate the waveforms and charge pump activation for the stimulation application.

Referring to FIGS. 4A and 4B, systems have become available that are more portable than those of the type shown in FIG. 2. With such systems, a portable generator which an LCD screen contains a microcontroller configured to permit control over electro-stimulation of associated electrodes (pads). The commercial device shown in FIG. 4A is but one example. An exemplary circuit diagram for the programmable energy generator is shown in FIG. 4B, where the system 1 comprises a portable control system 2 configured to deliver power signals to the electrodes (pads) 3a and 3b through cables 4a and 4b, respectively, in order to generate energy to the muscles to which the pads are applied. The controller 2 typically includes a microcontroller 5, a power circuit 6, a battery recharge circuit 7 and an LCD screen 8. The left portion of FIG. 4B reflects a more detailed electrical schematic of the microcontroller 5, the power circuit 6, in this case a charge pump circuit, the battery recharge circuit 7 and the LCD screen 8. With this arrangement, electro-stimulation can be applied through electrodes “J1 CON1” and “J2 CON2” within the charge pump circuit 6.

Given the ubiquity of smart mobile consumer electronic devices, it seems that a combination of portability and accessibility is needed. In that regard, those people most likely to encounter muscle spasms and other types of muscle injuries are also those likely to carry a smart mobile device. Embodiments of the present invention bring immediate muscle relief to athletes without the need of carrying with them an electro-stimulation system.

### SUMMARY

In that regard, in one embodiment, a system for facilitating muscle therapy is provided, where the system comprises a mobile device configured to download a programmable configured to generate waveform signals that can be employed by a power circuit to generate and deliver energy conforming to the signals, to a muscle pad. The embodiment may further comprise the power circuit configured to generate energy conforming to the signals generated by the downloadable application, where the power circuit is further configured to deliver such energy to the muscle pad.

In another embodiment, a system such as that described above may further comprise one or more muscle pads configured to be attached to a patient's muscle and configured to apply energy generated and delivered by the power circuit. Of course, the present technology is non-invasive, so application to a patient's muscle pragmatically means applying the pad to the skin of the patient proximal to the muscle desired to be treated. In one embodiment, the muscle pad comprises conductive adhesive support tape and conductive gel. In other embodiments, the muscle pad includes circuitry and/or controls adding a certain amount of intelligence to permit communication with the application within the mobile device.

It is contemplated that in some cases the waveform signals are generated based upon muscle metric feedback. In some particular cases, the waveform signals are generated based upon a corollary (of which there may be more than one) between the muscle metric feedback and the amount of energy delivered, whether manually or automatically, where the corollary may comprise empirical data generated by clinician experience, empirical data generated by research, a table of information readily available to the clinician, an algorithm reflecting a corollary. Indeed, in one or more automated embodiments, the waveform signals are generated automatically based upon a pre-established corollary stored so as to be accessible by the downloadable application when generating the waveform signals. The pre-established corollary may comprises a table and/or algorithm stored within the downloadable application, or stored elsewhere and added after downloading of the application. In one embodiment, at least one metric comprises muscle impedance. If desired, embodiments may comprise a muscle metric feedback circuit configured to detect at least one muscle metric in the form of feedback usable by the downloadable application to generate appropriate waveform signals, wherein at least one muscle metric is muscle impedance. Other metrics may be used, of course, including but not limited to transmission loss measurement, conductivity, water content, tissue damage, pain indicators, etc.

In some embodiments, the power circuit may reside at least in part in the mobile device. In others, the power circuit may reside at least in part in the muscle pad. Or it may reside entirely within the mobile device or the muscle pad. Likewise, the metric feedback circuit may reside at least in part in the mobile device, at least in part in the muscle pad, or entirely within one or the other. It is contemplated that some embodiments may further comprise a discrete component, wherein the discrete component is configured to be in electrical communication with the mobile device and the muscle pad, and where the discrete component may house at least a part of the power circuit, at least a part of the muscle metric feedback circuit, or the entirety of one or both circuits. The discrete device may be in wired communication with the mobile device and/or the muscle pad or in wireless communication with one and/or the other.

It is contemplated that the waveform signals may comprise one or more of numerous possible configurations. For example, the waveforms signals comprise one or more of either a pulse train, a pulse, a sinusoidal wave, a triangle wave, a square wave, or an amplitude modulated wave.

In another embodiment of the invention, an application downloadable to a mobile device is provided for facilitating muscle therapy, where the application is programmed and configurable to generate waveform signals configured to be employed by a power circuit to generate energy conforming to the signals to a muscle pad. If desired, a system may be provided comprising the downloadable application, where the system further comprising a muscle pad configured to be attached to a patient's muscle (i.e., the skin in proximity to the muscle to be treated) and configured to apply energy generated by a power circuit configured to receive waveform signals generated by the application downloaded to the mobile device.

### BRIEF DESCRIPTION OF THE FIGURES

The detailed description of some embodiments of the invention will be is made below with reference to the accompanying figures, wherein like numerals represent corresponding parts of the figures.

FIG. 1 is a photograph showing examples of electro-stimulation electrodes (or pads) used for muscle therapy;

FIG. 2 is a photograph showing similar examples of electro-stimulation electrodes (or pads) used for muscle therapy coupled to an electro-surgical generator;

FIG. 3 is a schematic view of how signals generated by electro-stimulation systems translates into muscle elongation and contraction;

FIG. 4A is a photograph of one example of a prior art portable electro-stimulation device employing a microcontroller;

FIG. 4B is a schematic diagram showing an example of the components and electrical circuits within a prior art portable electro-stimulation device employing a microcontroller;

FIG. 5 is a schematic view of one embodiment of the present invention;

FIG. 6 is a schematic view of an alternative embodiment of the present invention;

FIG. 7 is a schematic view of an alternative embodiment of the present invention;

FIG. 8 is a schematic view of an alternative embodiment of the present invention;

FIG. 9 is a schematic view of an alternative embodiment of the present invention;

FIG. 10 is a schematic view of an alternative embodiment of the present Invention;

FIG. 11 is a schematic view of an alternative embodiment of the present invention.

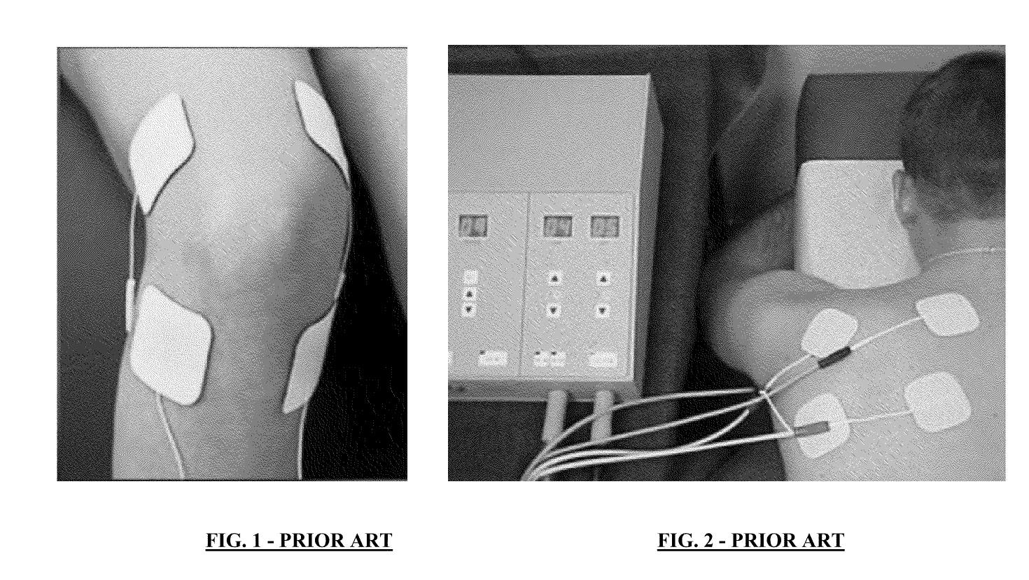

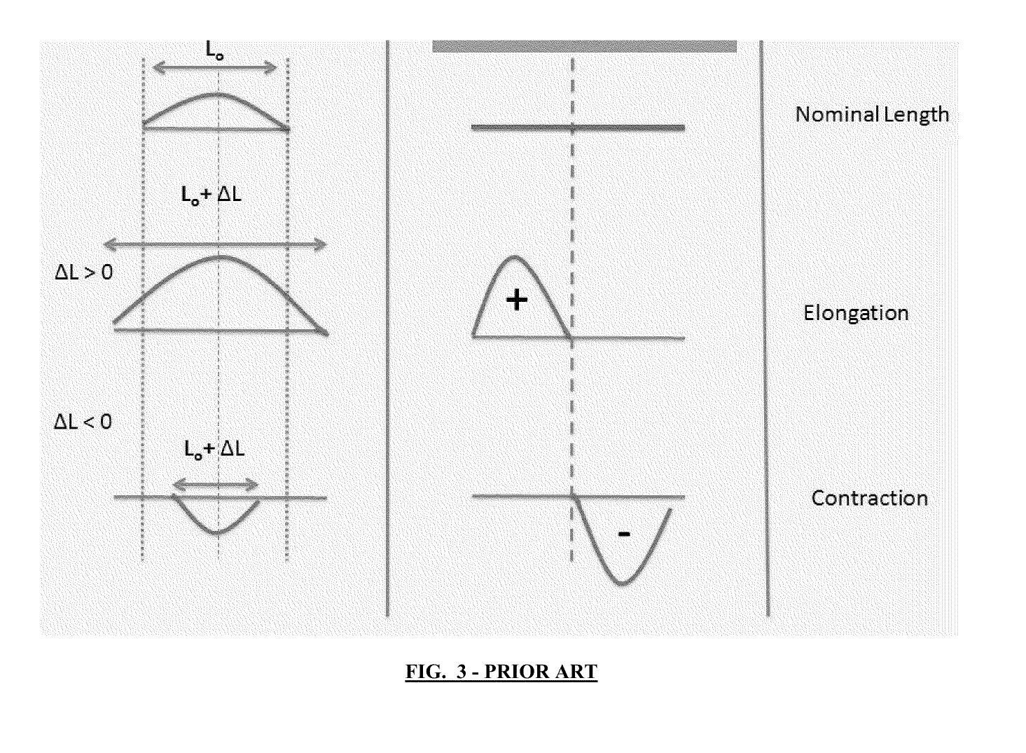

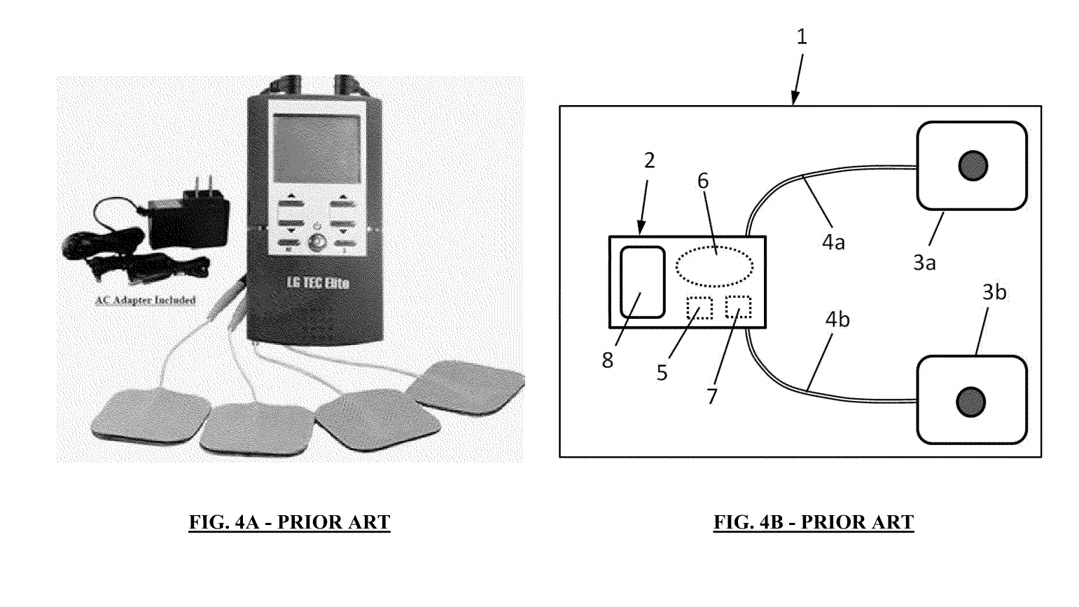

### DETAILED DESCRIPTION OF CERTAIN EMBODIMENTS

Although there are numerous companies with electro-stimulation products being sold, there are no systems utilizing the computing control power and graphic user interface of a mobile device, including tablets, notebooks and smart phones such as the iPhone® or Android® brand mobile devices (by example only), as well as numerous other examples of mobile devices with certain computing and trans-ceiving capabilities (capable of transmitting and receiving communications/signals). Using a mobile device, such as a smart phone, waveform outputs can be generated or triggered in conjunction with a charge pump circuit, within or without the mobile device, to meet the needs of the user utilizing a graphical user interface.

By way of explanation, a smart mobile device may be configured to perform calculations, such as power delivered, using the voltage and current waveform equations as shown in the derivations below:

V(t)=A sin(2πft+θ) (1)

I(t)=A sin(2πft+θ) (2)

where V(t) is the voltage, I(t) is the current, A is the sinusoid voltage or current amplitude, f is the waveform frequency, and θ is its phase angle. The power may be calculated through Equation (3) below and its reading is utilized by the user to ascertain treatment settings and effectiveness.

Power=|V(t)∥I(t)|PF (3)

where PF is the power factor, with the cosine of θ (θ=the power phase angle between the voltage and current) being one example of the power factor. Similar logic applies to other waveform types, high and low frequency.

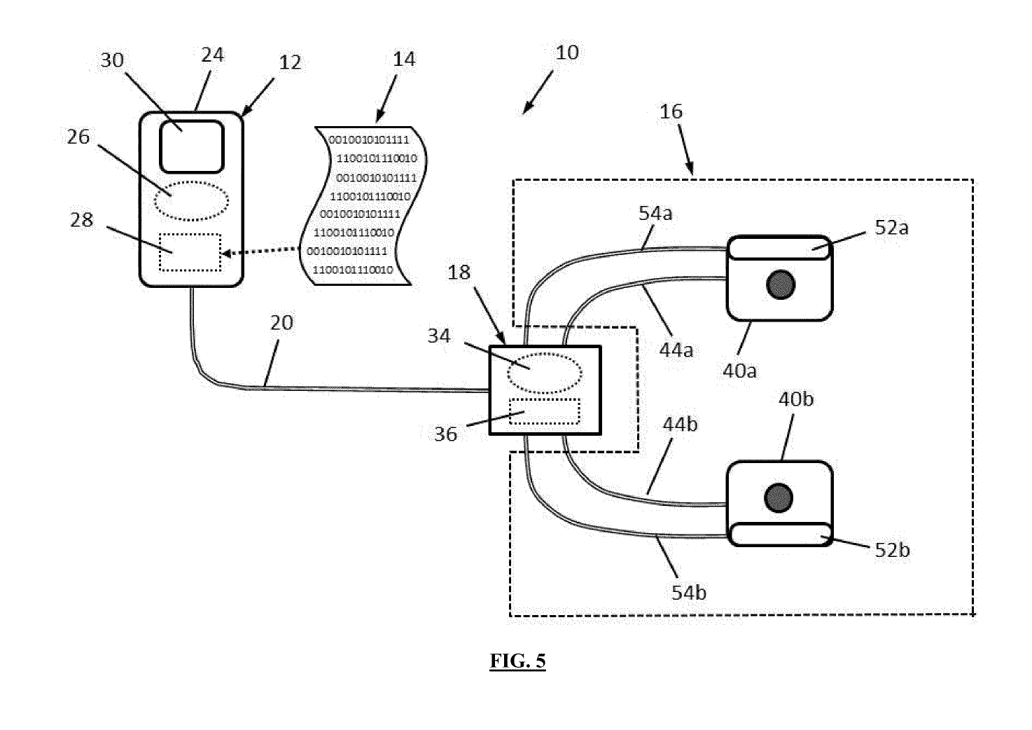

FIGS. 5 through 11 show various embodiments of the present invention, by example. Generally, the electro-stimulation systems of the present invention comprises a mobile device configured with a downloadable application written specifically to interface with one or more electrodes connected directly or indirectly to the mobile device using a compatible sync cable or wirelessly. For example, an iPhone® could be connected using an Apple® sync cable. For Android® devices, a USB 2.0 cable connection may be utilized. Handshaking is necessarily provided for in the various embodiments of the system to permit communication therebetween. Most mobile devices, including the iPhone® generates more battery power than the typical CR2025 watch battery and may be charged while being used for therapy.

Embodiments of the invention herein may also be employed to control an off-the-shelf electro-stimulation unit. A screen showing a graphic user interface screen can employed by the patient during treatment to control the energy to be delivered to the muscles. The downloadable smart device application preferably is configured to permit the smart device to control the treatment options, including waveform generation, power, duty cycle, time of treatment, frequency and/or monitoring of feedback to name a few examples.

Advantageously, electro-stimulation may be delivered and controlled while the user is performing other functions with the mobile device, including making and receiving calls, listening to music, watching movies, and/or working with other apps. Embodiments of the system employ a plug in a charger connection adapter to the electro-stimulation cable (if connected through the sync cable) that can be used to maintain mobile device charge life. If the unit is operating in wireless mode, a user may plug the mobile device into the charger device without interference to the electro-stimulation or multitasked smart device operation.

### Feedback and Performance Metrics

Given that electro-stimulation is delivering voltage and current to the muscle site, a feedback signal based off body impedance, power phase angle, etc., can be provided back to the smart phone system. This information can be used to determine treatment effectiveness, improvement status, or muscular efficiency etc so the user can ascertain their status in the treatment/improvement process. Similar techniques are used with electro-surgical equipment producing modified power curves based on frequency dependent impedance profiles, or within ultrasonic medical instrumentation adjusting acoustic power based on impedance and phase.

Using feedback, the smart phone can produce a gauge for the user to assist them in determining treatment effectiveness and/or athletic status for upcoming activity. For instance, a gauge metric labeled “Endurance Modulus” could be used, utilizing the impedance profile with an elasticity look up table for muscles with measured lactic acid levels. A scale from 1 to 10 (the high level indicating peak effectiveness), can be displayed providing a physical therapy decision information for treatment.

### Graphic User Interface

The graphic user interface will allow a user to program any setting to remedy the pain or muscle tone a region. A survey of parameters such as application time, power, or duty cycle can be provided to the user for selected choice of optimal recovery. Physical therapist or doctors can prescribe treatment prescriptions by uploading output parameters in the devices memory or with downloadable email data files that are read by the device's application that was downloaded from an application website. For other users not associated with a physical therapist or doctor, a default setting can be used. Downloadable applications are used to provide mobile devices such as smart phones with the controlling logic to drive output into electrode patches.

Referring to FIG. 5, specific embodiments may be described in detail. In that regard, one exemplary system 10 comprises a mobile device 12 configured to download an application program 14 permitting interface between the mobile device 12 an array of controllable electrodes 16. In one embodiment, a discrete component 18 is employed between the array of controllable electrodes 16 and the mobile device and connected to the mobile device via a sync cable 20 (or wirelessly in alternative embodiments). It is contemplated, and therefore should be understood by a person of ordinary skill in the art, that most if not all of the embodiments described by example herein may comprise a plurality of components linked electrically in wired and wireless formats. So although one particular embodiment may be described as comprising a sync cable, wireless communication may be employed in lieu of the cable or as an available alternative means of communication (i.e., both wired and wireless formats may be available within the same embodiment).

In the embodiment of FIG. 5, by example, the mobile device, which could be one of numerous intelligent (i.e., computerized) consumer multi-task electronic devices, such as those sold by Apple®, Motorola®, Samsung®, LG®, etc. As indicated above, they are ubiquitous amongst populations around the world. What is important about such smart devices, is they have existing technology within that permits ready adaptation to muscle electro-stimulation therapy by downloading a program 14 that is written to be controllable by the existing components, or with supplementation with additional components and/or features that can be added post-market or manufactured together initially. In that regard, a typical mobile device 12 comprises a housing enclosing a microcontroller (microprocessor) 26 and memory 28, along with circuitry designed to permit control of functionality inherent in the device or in the operation and performance of applications downloaded to the device. The mobile device is also typically configured so as to permit a user to configure certain features and functionality to optimize performance or personalize operation. The downloadable application 14 is configured to employ the existing technology within an existing mobile device and to configure operation to interface with a number of possible arrangements of electro-stimulation systems, whether custom or off-the-shelf varieties. The application may also be configured to employ circuitry and/or firmware that is added to the mobile device after-market or incorporated within the mobile device initially.

As with most smart mobile devices, a visible screen 30 is included, such as an LCD screen. Most recently, they are touch-screen capable, but need not be in order to effectively carry out the functionality of the embodiments described herein. The mobile device may include other modes of control, including buttons, scroll wheels, etc., where touch-screen capability does not exist. In either case, the downloaded application 14 is configured to accommodate one or the other or both.

In one embodiment of the system, the discrete component 18 is configured to house at least in part a power circuit 34 and, if so desired, a feedback circuit 36. Where one or both are employed, they are electrically interfaced with the microcontroller 26 to permit user control over the amount of energy delivered and/or adjustment in the power signals generated based upon feedback. The component 18 may be configured to plug directly into the mobile device 12 or be electrically connected to the mobile device with cable 20 or wirelessly. The power circuit 34 may be configured to interface with a part of the existing circuitry in the mobile device if so desired to optimize power output, when controlled by the microcontroller 26 in use by a consumer. Preferably, the discrete component 18 is configured with ports to permit wired interface with the array of controllable electrodes 16, or in some embodiments, wirelessly interface with the electrodes 16. It should be noted that the system is preferably configured to accommodate a single electrode at a time if that is all that is desired by the user, but the embodiments preferably accommodate a plurality to maximize use across numerous scopes of muscle therapies (large and small muscle areas).

In one arrangement of an embodiment of the present invention, the array of electrodes may comprise a first electrode (pad) 40a and a second electrode (pad) 40b, each respectively connected to the power circuit 34 of the discrete component 18 via cables 44a and 44b. Where feedback is desired for manual and/or automated modulation of energy delivery, one or more sensors may be employed directly or indirectly connected to the electrode pads 40a, 40b, via cables 54a, 54b, respectively, or wirelessly. As indicated above, one or more metrics of feedback may be detected and transmitted through the feedback circuit 36 and microcontroller 26, including impedance and other physiological and/or patient responses. Embodiments of the invention are preferably configured to be manually controlled simply by the clinician or patient desiring to module the energy delivery based upon empirical, visual or other sensory feedback detected by the user and/or clinician. For example, a user may visually sense unusual color tone response in the skin surrounding the muscle and desire to adjust energy delivery accordingly. It should also be noted that, because signals may be delivered in one of numerous forms, including pulsed and continuous formats, that energy delivery may be controlled by pre-set times programmed into the system, or by manual adjustment of energy delivery duration.

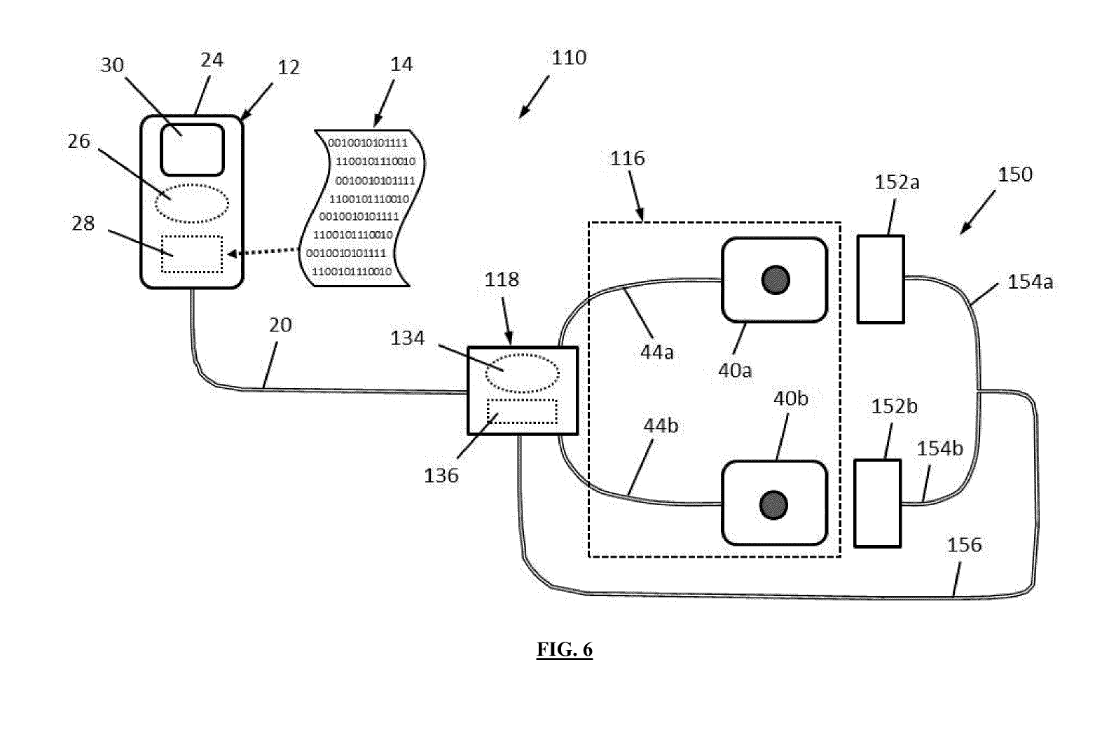

It should be noted that all or part of the power circuits and/or feedback circuits may be housed with the discrete component 18, where some parts of either or both circuits reside within the mobile device and/or the electrode pads. It should also be noted that the sensors 52a and 52b may be in the form of resistors placed between the positive and negative wires on the electrode cables 44a, 44b in some cases, or embodied within or without the electrode pads 40a, 40b. For example, with reference to FIG. 6, an independent sensor array may be employed where the array of electrode pads 16 does not include feedback sensors. In that regard, embodiment 110 may comprise the same or similar mobile device 12, and the same or similar downloadable application 14. But the array of electrodes 116 may be simply one or more electrodes connectable wired or wirelessly to a discrete component 118 configured to house all of part of a power circuit 134 and, optionally, all of part of a feedback circuit 136, where feedback may be provided by a separate feedback sensor array 150. Such sensory array may comprise one or more sensors 152a, 152b configured to be applied to the area of the muscle(s) being stimulated or treated, and connected to the feedback circuit 136 via cables 154a, 154b, respectively for each sensor 152a, 152b, and optionally a central cable 156 leading to the discrete component 118. Of course, the sensor signals may be wirelessly conveyed to the discrete component 118. This may be configured as a plug and play sensor array, meaning that one or more sensor arrays may be employed with the discrete component 118 where interface is more universal in format, or the array may be manufactured tailored to a particular discrete component to preclude employment of competing brands of sensor arrays. As with the exemplary embodiments of FIG. 5, the power circuit and optional feedback circuit are interfaced with the controller 26 of the mobile device for effective control of operation.

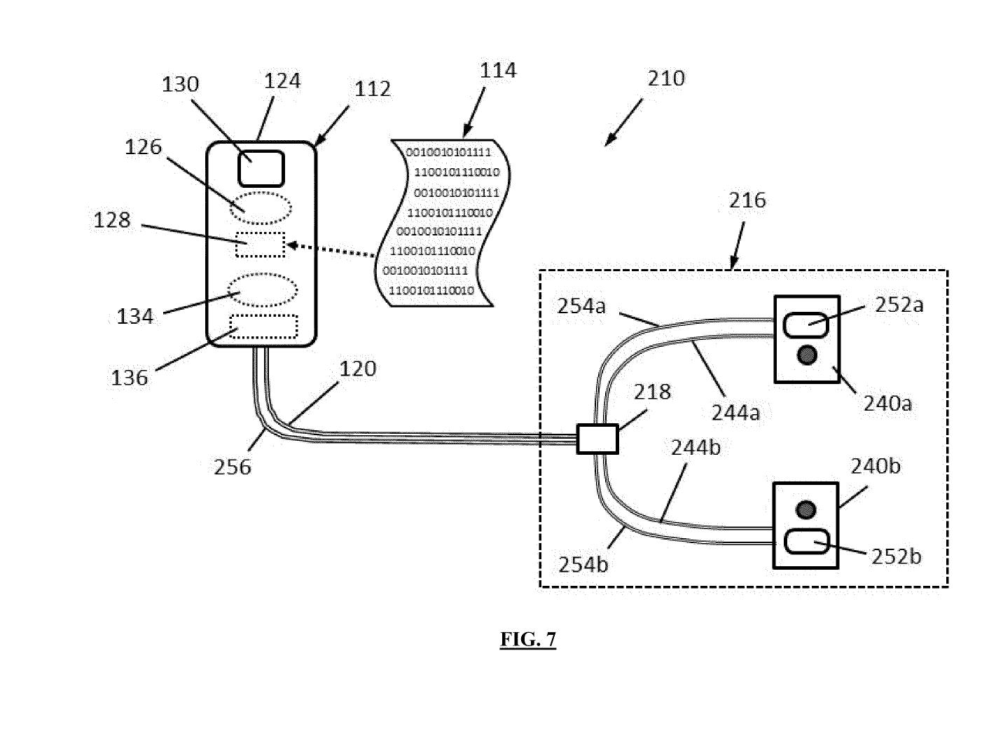

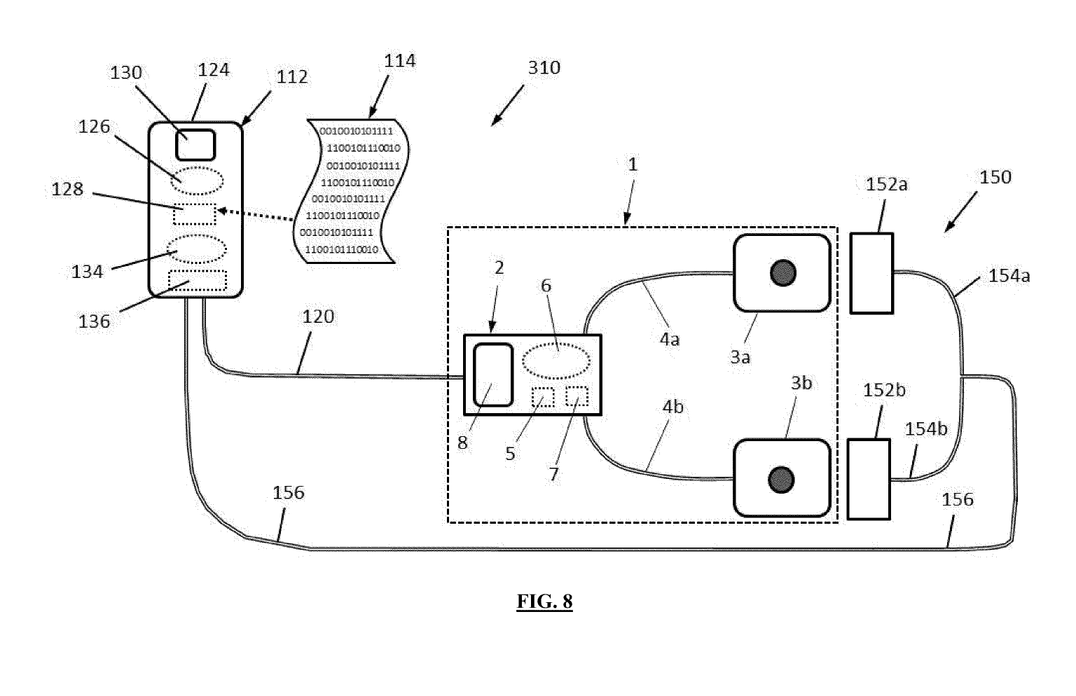

As a person of ordinary skill in the art can appreciate, a variety of arrangements are possible with the invention herein. Indeed, with reference to FIGS. 7 and 8, in embodiments 210 and 310, the mobile device 112 may comprise a housing enclosing a microcontroller 126, memory 128, all of part of a power circuit 134 and, optionally, all of part of a feedback circuit 136. As indicated above, the power circuit and feedback circuit may be added after market or built together originally with the mobile device. In either case, the downloadable application 114 is configured to accommodate the location of the power and feedback circuits being housed within the mobile device. Of course, the application 14 may also be so configured and usable with mobile device 12 or mobile device 112. Likewise for downloadable application 114. In either case, both are configurable to accommodate a variety of possible electrode array arrangements and sensor array arrangements.

For embodiment 210 of FIG. 7, an electrode/sensor array 216 may comprise a pre-packaged or later assembled system of electrodes 240a, 240b, connected with cables 244a, 244b, respectively to component (e.g., junction box) 218 that is configured to accommodate electrically interface with the controller 126 and power circuit 134 of mobile device 112 through cable 120 or wirelessly. Likewise, the array 216 may comprise a pre-packaged or later assembled system of sensors 252a, 252b connected with cables 254a, 254b, respectively to component 218 for interface with the controller 126 and feedback circuit 136 of mobile device 112 through cable 256 of wirelessly. Once again, the electrode/sensor array may be configured as a plug and play arrangement with the mobile device, or be configured to be tailored specific to one or more specific mobile devices. Such an arrangement allows both pre-assembled kits and/or modularity to the embodiments of the invention herein. For example, one might market the downloadable application on memory media (or from a website on-line) combined with an electrode array and/or a feedback array, and also with a discrete component 18, 118. Such “kits” may be sold so as to be usable with one or more existing or future developed mobile devices, or be configured to be limited for use with one or more specific mobile devices having certain features or restrictions. Or a “kit” may be sold that includes an entire system, including the a mobile device. the discrete component, a discrete electrode array, a discrete sensor array or a combined electrode/sensor array, all or some parts configured for plug and play arrangement or cabled or wireless interface. Some modularity may be sold as part of a package that can be combined with other components purchased off-the-shelf by the same or other manufacturer.

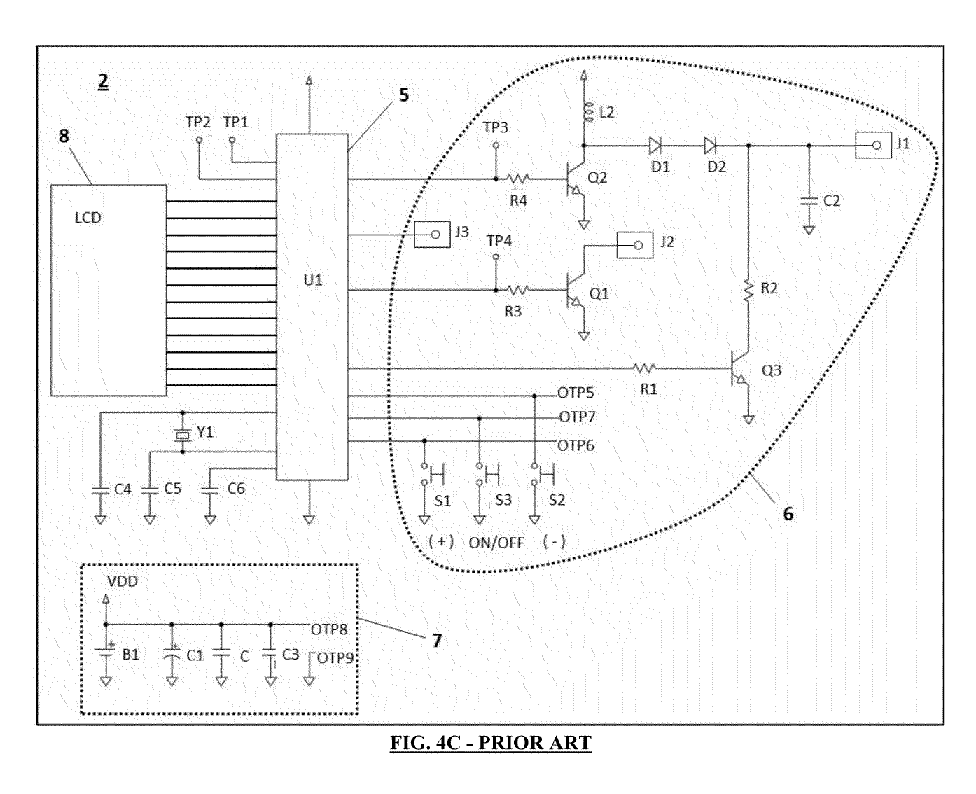

A further example of modularity is illustrated by example in FIG. 8, where the mobile device 112 and downloadable application 114 may be combined with a prior art electrode pad array 1 (as described above in association with FIGS. 4A and 4B), to which may be added the modular sensor array package 150 described above in association with FIG. 6. In this case, by example, the sensory array is connected directly via central cable 156 to the mobile device 112 or wirelessly, rather than interfacing with a discrete component 118, as in the embodiment of FIG. 6. The application 114 is configured preferably to permit the controller 126 and screen 130 to override the existing controller 5 and screen 8 of the off-the-shelf prior art electrode array system 1. Redundancy may be maintained if so desired, but probably not necessary.

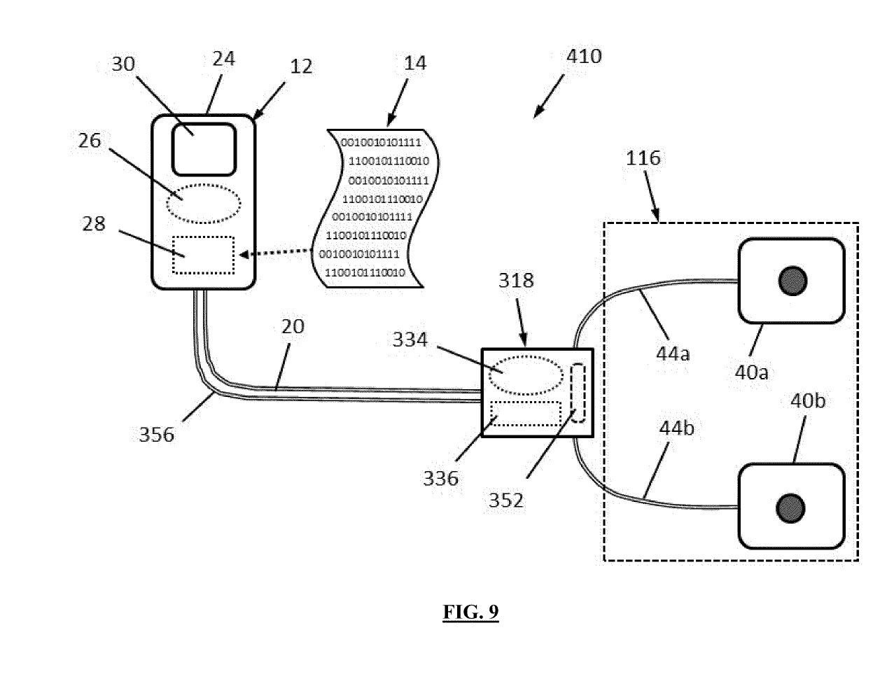

Further variations may be appreciated with reference to FIGS. 9, 10 and 11, where various arrangements of feedback circuits and sensors may be employed. In the embodiment 410 of FIG. 9, the mobile device 12 interfaces with a discrete component 318 that houses not only all of part of a power circuit 334 and feedback circuit 336, but also a sensor 352. The discrete component 318 is preferably configured to be electrically linked to the mobile device via cable 20 (or wirelessly) for power/energy control and via cable 356 (or wirelessly) for feedback control. Electrode array 116, like the embodiment of FIG. 6, reflects a more basic array of one or more electrodes 40a, 40b connected via electrode cables 44a, 44b, respectively or wirelessly. It should be noted that while the embodiments herein are shown with two electrode pads, embodiments of the invention may be employed with a single electrode pad or three or more electrode pads. Indeed, it is also contemplated that multiple “sets” of electrode and sensory arrays may be employed for different parts of the body, each directly interfacing with a mobile device or indirectly interfacing with a mobile device through a discrete component, again in plug and play fashion or customized fashion.

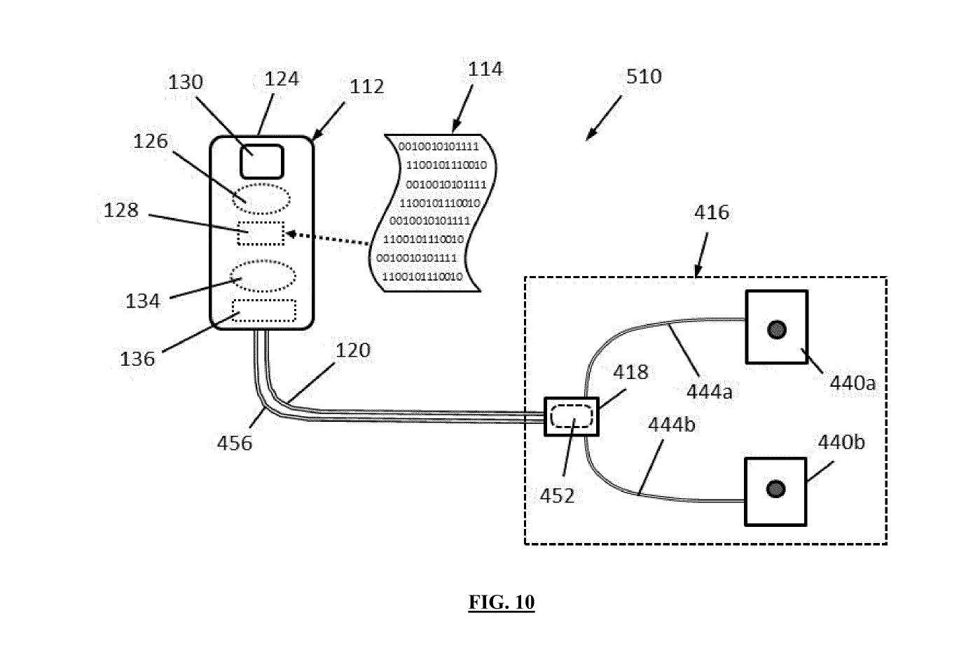

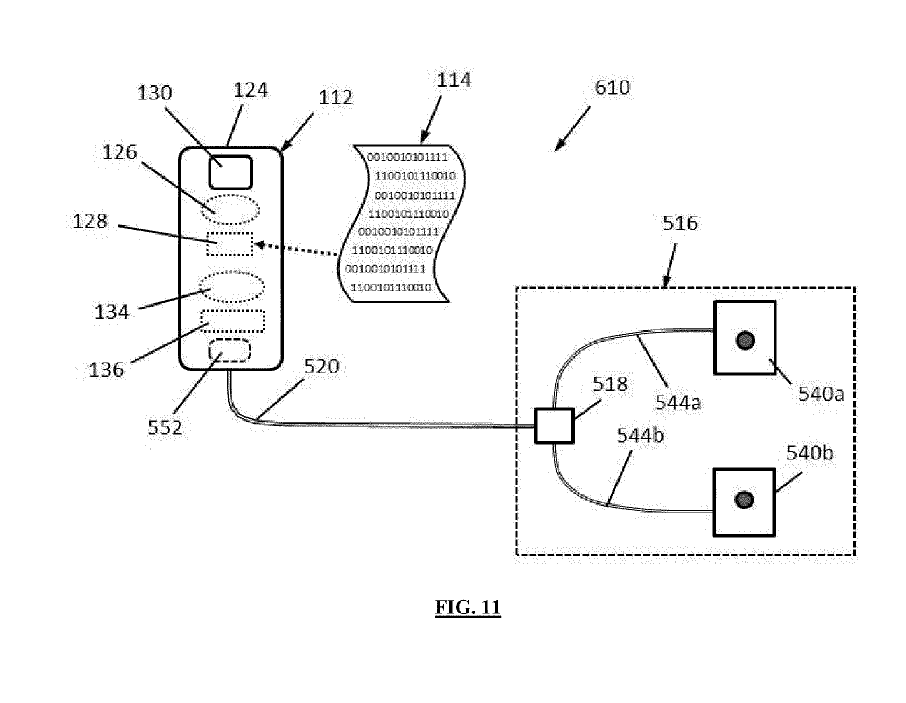

With the embodiment of FIG. 10, by example, an electrode/sensor array 416 comprises an array of electrode pads 440a, 440b connected to discrete component 418 via cables 444a, 44b, respectively (or wirelessly), where the discrete component 418 comprises a feedback circuit 452 connected via cable 456 (or wirelessly) to the mobile device 112, which comprises at least a part of the entirety of a power circuit and a feedback circuit. The embodiment of FIG. 11 shows by example yet another arrangement where the electrode array 516 comprises electrode pads 540a, 540b, connected to discrete component (e.g., junction box) 518 via cables 544a, 544b (or wirelessly). The discrete component 518 is thereby synced with the mobile device 112 via cable 520 or wirelessly.

It should be noted that feedback control may be monitored through the interfaces to initiate decision logic within the communication device to control adjustment in the delivered output. Such feedback control leads to other applications using embodiments of he present invention such as the following:
Portable electro-surgical units
Portable ultrasonic generators
Release of insulin for diabetic patients with closed loop monitoring and dosage delivery
Stimulation feedback toys or tools using the smart phone interface (Adult and Children)
Video game feedback in which the Microsoft Xbox 360 or Sony PS3 transmitter/receiver unit are programmed to communicate wirelessly with the smart phone to produce an electro-stimulus to the player to mimic game play action, or
Remote control feedback stimulation for robotic surgical devices, to list just a few examples.

Thus, persons of ordinary skill in the art may appreciate that numerous design configurations may be possible to enjoy the functional benefits of the inventive systems. Thus, given the wide variety of configurations and arrangements of embodiments of the present invention the scope of the invention is reflected by the breadth of the claims below rather than narrowed by the embodiments described above.

What is claimed is:

1. A system for facilitating muscle therapy, the system comprising a mobile device comprising a programmable downloadable application to generate waveform signals, the waveform signals configured to be employed by a power circuit to generate and deliver energy conforming to the signals to a muscle pad.

2. The system of claim 1, further comprising a power circuit configured to generate energy conforming to the signals generated by the application downloaded to the mobile device, the power circuit further configured to deliver such energy to the muscle pad.

3. The system of claim 2, further comprising a muscle pad configured to be attached to a patient's muscle and configured to apply energy generated and delivered by the power circuit.

4. The system of claim 3, wherein the muscle pad comprises conductive adhesive support tape and conductive gel.

5. The system of claim 1, wherein the waveform signals are generated based upon muscle metric feedback.

6. The system of claim 5, wherein the waveform signals are generated based upon a corollary between the muscle metric feedback and the amount of energy delivered.

7. The system of claim 6, wherein the waveform signals are generated automatically based upon a pre-established corollary stored so as to be accessible by the downloadable application when generating the waveform signals.

8. The system of claim 5, wherein at least one metric is muscle impedance.

9. The system of claim 5, further comprising a muscle metric feedback circuit configured to detect at least one muscle metric in the form of feedback usable by the downloadable application to generate appropriate waveform signals, wherein at least one muscle metric is muscle impedance.

10. The system of claim 3, wherein the power circuit resides at least in part in the mobile device.

11. The system of claim 3, wherein the power circuit resides at least in part in the muscle pad.

12. The system of claim 9, wherein the muscle metric feedback circuit resides at least in part in the mobile device.

13. The system of claim 9, wherein the muscle metric feedback circuit resides at least in part in the muscle pad.

14. The system of claim 3, further comprising a discrete component, wherein the discrete component is configured to be in electrical communication with the mobile device and the muscle pad.

15. The system of claim 14, wherein the discrete component houses at least a part of the power circuit.

16. The system of claim 14, wherein the discrete component houses at least a part of the muscle metric feedback circuit.

17. The system of claim 14, wherein the discrete component is configured to communicate wirelessly with either or both of the mobile device and the muscle pad.

18. The system of claim 1, wherein the waveforms signals comprise one or more of either a pulse train wave, a pulse wave, a sinusoidal wave, a triangle wave, a square wave, or an amplitude modulated wave.

19. An application downloadable to a mobile device for facilitating muscle therapy, the applicable programmed and configurable to generate waveform signals, the waveform signals configured to be employed by a power circuit to generate energy, conforming to the signals, to a muscle pad.

20. A system comprising the downloadable application of claim 17, the system further comprising a muscle pad configured to be attached to a patient's muscle and configured to apply energy generated by a power circuit configured to receive waveform signals generated by the application downloaded to the mobile device.
